(19) United States
(12) Patent Application Publication
(10) Pub. No.: US 2007/0198075 A1
(43) Pub. Date: Aug. 23, 2007

(54) Bifurcated aneurysm treatment arrangement

(76) Inventor: Elad I. Levy, Amherst, NY (US)

Correspondence Address: Donald N. Halgren, 35 Central Street, Manchester, MA, 01944, US

(21) Appl. No.: 11/645,249

(22) Filed: Dec. 23, 2006

Related U.S. Application Data
(60) Provisional application No. 60/753,764, filed on Dec. 23, 2005.
(60) Provisional application No. 60/755,639, filed on Dec. 31, 2005.

(51) Int. Cl. A61F 2/84; A61F 2/94

(52) U.S. Cl. 623/1.11; 623/1.15; 623/1.36; 623/1.31

(57) ABSTRACT

A vascular aneurysm-treating stent arrangement having a proximal end and a distal end, the stent being formed of a differentially expandable material, wherein the distal end is deformably expandable to a cone shape. A deformable enclosed chamber is arranged on the distal end of the stent and nests within the aneurysm. A different embolic agent is introduced into the aneurysm and into the chamber.

### BACKGROUND OF THE INVENTION

1. Field of the Invention

This patent application for this invention relates to stent devices and their methods of use for treating cranial aneurysms. This non-provisional patent application is based upon Provisional Patent Application Ser. No. 60/753,764 filed Dec. 23, 2005, and upon Provisional Patent Application Ser. No. 60/755,639, filed December 2005, each of which are incorporated herein by reference.

2. Prior art

Current treatment of bifurcation aneurysms currently utilize balloons and a stent. However, such balloons may at least temporarily occlude blood flow through the vessels in which they are placed. Those balloons also need to be deflated and removed at the end of a vessel remodeling session. Such balloons may also rupture an aneurysm and/or a vessel when that balloon is inflated. Utilizing a stent with the balloon in a bifurcation aneurysm does not protect both of its efferent vessels. Such balloon vessel remodeling also requires two experienced surgeons and two catheters simultaneously, in a single vessel at the same time.

It is an object of the present invention to overcome the disadvantages of the prior art.

It is a further object of the present invention to provide a bifurcation aneurysm treatment which will allow proper blood flow during the treatment procedure, and to prevent reflux of any embolic agent placed within the aneurysm.

### BRIEF SUMMARY OF THE INVENTION

The present invention relates to an elongated aneurysm-treating stent device having an open proximal end, and an open distal end. The stent device is cylindrical and is preferably constructed from a woven pattern of metallic fibers. The proximal end of the stent device may have a plurality of radio opaque markers thereon. An elongated electrolytic tethering wire is arranged at several circumferential locations on the proximal end of the stent device. Those tethering wires join a common electrolytic tethering wire which extends through a delivery catheter. The tethering wires are attached to the proximal end of the stent device at electrolytic junctions. Those electrolytic junctions are arranged so as to be severed once the stent device has been put in place. The web design of the stent device is woven so as to have larger openings between the web fibers towards the distalmost end of the stent device.

A generally hemispherically-shaped “drip” chamber is fixably attached to the distalmost end of the stent device. The distalmost chamber has a floor section extending thereacross which effectively closes off the distal end of the stent device. The hemispherical chamber and the floor thereacross are entirely preferably radiopaque. The woven nature of the chamber provides smaller cell sizes between adjacent wires or fibers, comprising the chamber. Those cell or opening sizes within the chamber and the chamber floor are however, wide enough to admit a 0.014 or 0.010 microcatheter therethrough.

Both the drip chamber and the body of the stent device may be made of self-expanding metal, such as nitinol or expandable stainless steel or the like. Such material may also be plated with for example, a gold or platinum thereon. Such metal or plating also, may be porous, so as to carry and emit drugs therefrom, upon their delivery into a body vessel.

The body of the stent device as well as the drip chamber are expandable, for example from a 3 millimeter diameter to about a 10 millimeter diameter to permit it to fit within the parent vessel and also then to expand to nest within the aneurysm neck.

The stent device is arranged so that the weave of the fibrous metal adjacent its distalmost end expands more widely than that at a proximal position, so as to create and generate an outwardly tapered “waffle cone” shape, which would provide wide enough cell openings in the weave disposed between the efferent vessels for blood to flow therethrough. The diameter of the chamber floor at the proximal end of the drip chamber is designed so as to nestingly mate with the approximate diameter of the neck of the aneurysm itself. The drip chamber itself is arranged to expand to a diameter larger than the diameter of the neck of the aneurysm so as to permit a blocking nesting engagement therewith. The cell structure between the fibers of the woven drip chamber are smaller than the openings at the expanded distalmost end of the stent device itself. Those fibers are woven so as to effect such a trumpet or waffle cone shaped configuration to the distalmost third or quarter of the stent device. With such tapered expansion of those fibers, the open cell structure is inherently permitted to let blood pas therethrough, while also effecting the locking of the drip chamber within the neck of the aneurysm.

The introduction of an aneurysm treating stent device into an aneurysm is done by threading a microcatheter, bearing a micro wire, through the vasculature of the patient and into the bifurcation aneurysm. The microcatheter or sheath surrounding the microwire is pulled proximally, so as to leave the bare microware juxtaposed within the aneurysm itself. A catheter with a waffle cone stent device loaded therewithin is threaded over the microwire and that catheter is advanced into the aneurysm through the parent vessel thereadjacent. The microwire or guidewire is then removed by its withdrawal proximally through the delivery catheter. The waffle cone stent device with its attendant distalmost drip chamber thereon, in its unexpanded state, is guided through that parent vessel with the drip chamber disposed nestingly at the neck of the aneurysm. Withdrawal of the delivery catheter from the outside of the waffle cone stent and drip chamber would permit their respective self-expansion to occur. Adjustments in the position of the drip chamber and the waffle cone stent device may be made by the tethering wire which is attached to the proximal most end of the stent device. Once the stent device and drip chamber are properly placed, the tethering wire may be electronically separated from the proximal end of the stent device.

Upon withdrawal of the delivery stent from the drip chamber portions of the stent device, an arrangement of folded struts may flare out to their own spring tension or self-expansion capabilities, to permit the drip chamber to be firmly anchored within the neck confines of the aneurysm.

A new microcatheter or guidewire may be advanced through the waffle cone stent and drip chamber after it has been placed. That microwire or guide wire would be arranged so as to extend through one of the open cells in the floor of the drip chamber and also through the outer cells of the drip chamber as well. A further new microcatheter would then be threaded over that microwire or guidewire which extends distally beyond the drip chamber. Once that new microcatheter is in place distally beyond the distalmost end of the drip chamber, that microwire or guidewire is withdrawn proximally therefrom.

The microcatheter then acts as an ejector, through which Onyx™, an embolic agent, which is injected into the aneurysm itself. The dome of the drip chamber having small cellular openings therein, acts as a protective shield to prevent the Onyx from reflux into the parent vessel adjacent the aneurysm. Once the Onyx embolic agent has filled the fundus of the aneurysm, that delivery catheter is removed. A further microwire or guidewire may then be inserted through the stent device and into the drip chamber through its floor. That microwire or guidewire would then be removed and the drip chamber itself filled with a second material, such as a more viscous Onyx, metallic coils, or for example a nitinol plug. Once the drip chamber is filled with the second embolic material, that drip chamber will block the original onyx from entering the parent vessel.

The microcatheters and electrolytic tether wires may then be removed from the stent device and the aneurysm remains filled with multiple embolic material, now generally harmless to the patient.

The invention thus comprises a vascular aneurysm treating stent arrangement having a proximal end and a distal end, the stent being formed of a differentially expandable material, wherein the distal end is deformably expandable to a cone shape, and a deformable enclosed chamber arranged on the distal end of the stent. The deformable chamber preferably has an expandable foraminous floor arranged thereon. The chamber preferably has wall portions with a smaller opening pattern arranged therethrough. The stent device is preferably comprised of a woven material. The stent preferably has a severable tether arranged in its proximal end.

The invention also comprises a method of treating a bifurcated aneurysm having a neck portion, into a body vessel, comprising one or more of the following steps: introducing a stent assembly into the body vessel, the stent having a body portion and a distal chamber on the body portion; inserting the chamber into the aneurysm; expanding the chamber to a known dimension and volume; introducing a first delivery catheter through the body portion of the stent and through the chamber and into the aneurysm; injecting an first embolic material into the aneurysm; removing the first delivery catheter from the aneurysm and introducing a second delivery catheter through the body portion of the stent and into the chamber; injecting a known quantity of a second embolic material into the chamber, completely filling the chamber; nesting the chamber within the neck portion of the aneurysm; opening a strut arrangement into the aneurysm to secure the chamber within the aneurysm; placing a floor in a proximal portion of the chamber to segregate the second embolic material from the body vessel; expanding a distal portion of the stent body into a cone shape; expanding the chamber into a known volume within the aneurysm simultaneously with the expansion of the stent body.

The invention may also comprise a vascular aneurysm treating stent arrangement having a proximal end and a distal end, the distal end having a larger pattern of openings therethrough than any sidewall openings at the proximal end, upon delivery thereof. The distal end of the stent preferably has an expandable web floor disposed thereacross, the floor having an expandable dome-like chamber thereon to permit a first embolytic material to be disposed outwardly thereof, and a second embolytic material to be separately retained within the dome-like chamber. The second material preferably comprises metal coils. The second material in the chamber preferably comprises a blocking component to the first embolytic material. The distal end of the stent has enlarged openings thereacross to permit blood flow across the distal end of the stent, and the distal end of the stent has an expandable, aneurysm-nesting chamber thereon to anchor the stent thereat. The distal end of the stent preferably includes a plurality or articulable struts arranged to spread radially outwardly radially adjacent the expandable chamber to further anchor the stent within the aneurysm.

### BRIEF DESCRIPTION OF THE DRAWINGS

The objects and advantages of the present invention will become more apparent when viewed in conjunction with the following drawings, in which:

FIG. 1 is a side elevational view of a stent device and its associated drip chamber thereon;

FIG. 2A is an exploded view of the distal portion of the stent device and its drip chamber therewith;

FIG. 2B shows a side representation of the weave of each of the distal chamber and stent device of the present invention;

FIG. 3 is a side elevational view of an expanded drip chamber on the distalmost end of a partially expanded stent device of the present invention;

FIG. 3A is a plan view of the floor of the expanded drip chamber shown in FIG. 3;

FIG. 4 is a representation of the present stent device in a waffle cone-like expansion with the drip chamber arranged within the neck of an aneurysm;

FIG. 5 is a representation of a microcatheter and microwire arranged within an aneurysm to initiate treatment thereof;

FIG. 6 is a view similar to FIG. 5, showing its microcatheter removed and the microwire or guidewire remaining in the aneurysm;

FIG. 7 shows a delivery catheter being slid over the guidewire within the aneurysm;

FIG. 8 is a representation of the stent device self-expanded to its waffle cone shape and the drip chamber expanded within the neck of the aneurysm and still attached to the delivery catheter and tether arrangement;

FIG. 9 shows a representation of a microcatheter arranged through the stent device and drip chamber for delivery of embolic agents within the fundus of the aneurysm;

FIG. 10 shows a representation of a microcatheter arranged within the drip chamber through the waffle stent device for further treatment of a material within that drip chamber;

FIG. 11 is a representation of that microdelivery catheter injecting a second embolic material within that drip chamber; and

FIG. 12 is a representation of the self-expanded waffle cone stent and drip chamber arranged within the branch of the vessels and within the neck of the aneurysm which has thus been treated.

### DETAILED DESCRIPTION OF THE PREFERRED EMBODIMENTS

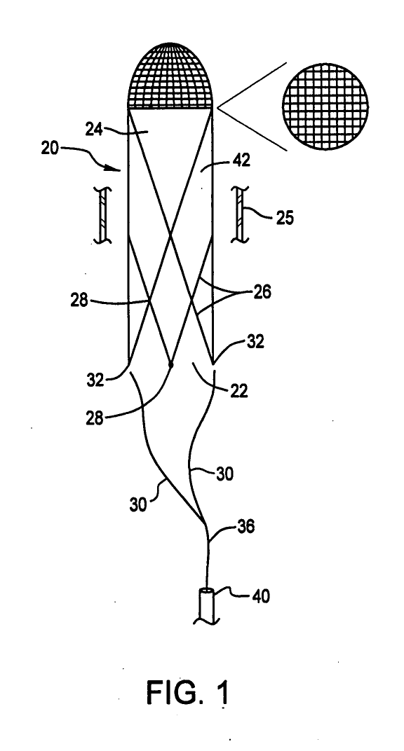

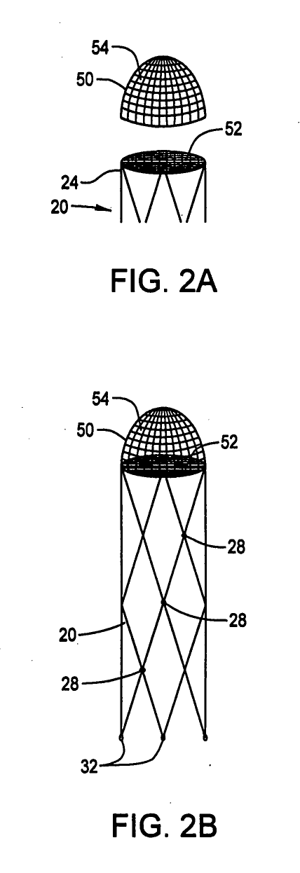

Referring now to the drawings in detail, and particularly to FIG. 1, there is shown the present invention which comprises an elongated aneurysm-treating stent device 20 having an open proximal end 22, and an open distal end 24. The stent device 20 is cylindrical and is preferably constructed from a woven pattern of metallic fibers 26. The proximal end 22 of the stent device 20 may have a plurality of arrayed, aligned, spaced apart radiopaque markers 28 thereon, as shown in FIGS. 1 and 2. A plurality of elongated electrolytic tethering wires 30, represented in FIG. 1, is arranged at several circumferentially arrayed electrolytic junctions 32 at spaced apart circumferential locations on the proximal end 22 of the stent device 20. Those tethering wires 30 join a common electrolytic tethering control wire 36 which extends through a delivery catheter 40. The tethering wires 32 are attached to the proximal end 22 of the stent device 20 at those electrolytic junctions 32. Those electrolytic junctions 32 are arranged so as to be severed by severing means, once the stent device 20 has been put in place in a body lumen 25. The web design of the stent device 20 is woven so as to have larger “cell” openings 42 between the web fibers 26 towards the distalmost end of the stent device.

A generally hemispherically-shaped “drip” chamber 50 is fixably attached to the distalmost end of the stent device 20, as shown in FIG. 2B, and as shown in an “exploded” view in FIG. 2A. The distalmost drip chamber 50 has a floor section 52 extending thereacross which effectively closes off the distal end 24 of the stent device 20. The hemispherically shaped chamber 50 and its attached floor 52 thereacross are preferably entirely radiopaque, and may be attached to the stent 20 as a separate material and separate woven construction from the stent 20 itself. The woven nature of the drip chamber 50 provides smaller cell sizes 54 between adjacent wires or fibers, comprising the drip chamber 50. Those cell or opening sizes within the chamber 50 and the chamber floor 52 are however, wide enough to admit a 0.014 or 0.010 microcatheter therethrough, as described hereinbelow.

Both the drip chamber 50, the chamber floor 52 and the body of the stent device 20 may be made of self-expanding memory metal, such as nitinol or expandable stainless steel or the like. Such material may also be plated with for example, a gold or platinum thereon. Such metal or plating also, may be porous, so as to carry, be re-supplied with (by subsequent re-coating with a separate drug delivery catheter) and to emit drugs therefrom, upon their delivery into a body vessel.

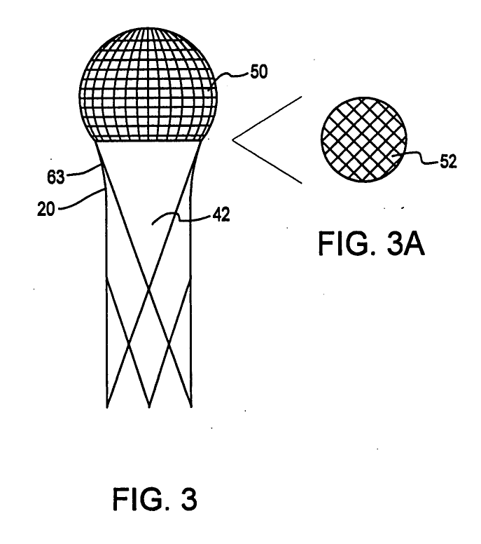

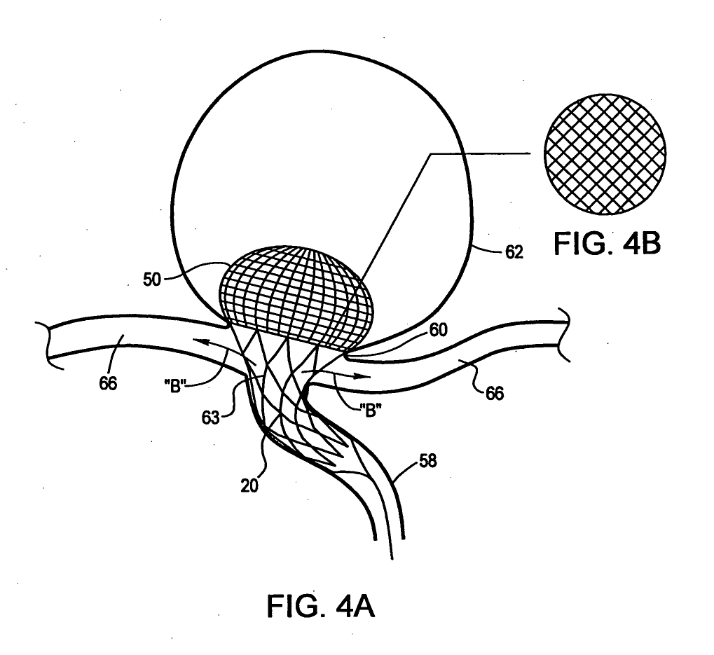

The body of the stent device 20 as well as the drip chamber 50 are expandable, as is represented in FIG. 3, for example from a 3 millimeter diameter to about a 10 millimeter diameter to permit it to fit within the parent vessel 58 and also then to “bulbously” expand beyond the diameter of the stent 20, so as to facilitate its “riesting” and anchoring within a neck 60 of an aneurysm 62, as represented for example, in FIG. 4.

The stent device 20 is arranged so that the weave of the fibrous metal adjacent its distalmost end expands more widely than that at a proximal position of the stent device 20, so as to create and generate an outwardly tapered “waffle cone” shape 63. Such distal conical expansion, for example, going from 3 mm to 10 mm, depending upon where it is constrained within the parent vessel and then expands in the aneurysm neck, the stent 20 would provide wide enough distally-enlarged cell openings in the weave disposed between the efferent vessels 66 for facilitating the blood “B” to flow therethrough. Such expansion is represented in FIGS. 3 and 4, and such blood flow is represented in FIG. 4. The diameter of the chamber floor 52 at the proximal end of the drip chamber 50 is designed so as to nestingly mate with the approximate diameter of the neck 60 of the aneurysm 62. The drip chamber 50 itself is arranged to expand to a diameter larger than the diameter of the neck 60 of the aneurysm 62 so as to permit a blocking nesting engagement therewith. The cell structure 54 between the fibers of the woven drip chamber 50 and floor 52 are smaller than the openings at the expanded distalmost end of the stent device 20. Those fibers 26 are woven so as to effect such a “trumpet” or “waffle cone” shaped configuration to the distalmost third or quarter of the stent device 20. With such tapered expansion of those fibers, the open cell structure is inherently permitted to let blood pas therethrough, while also effecting the locking of the drip chamber 50 within the neck 60 of the aneurysm 62. FIG. 3A represents the expanded nature of the floor 52 of the chamber 50.

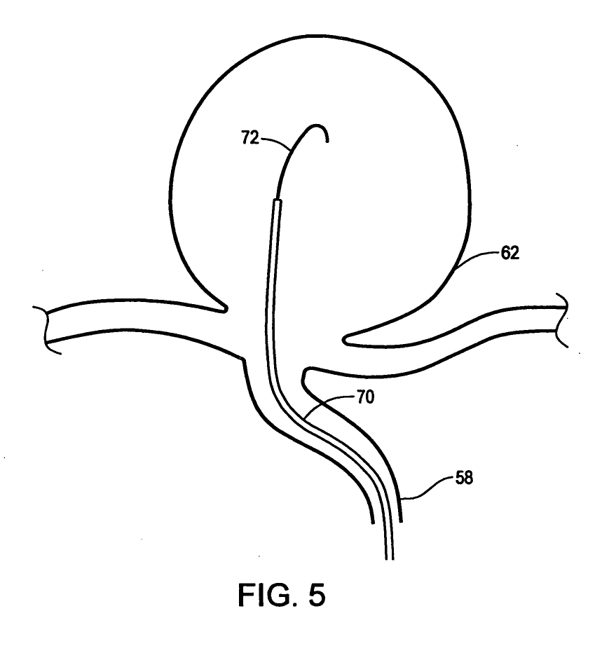

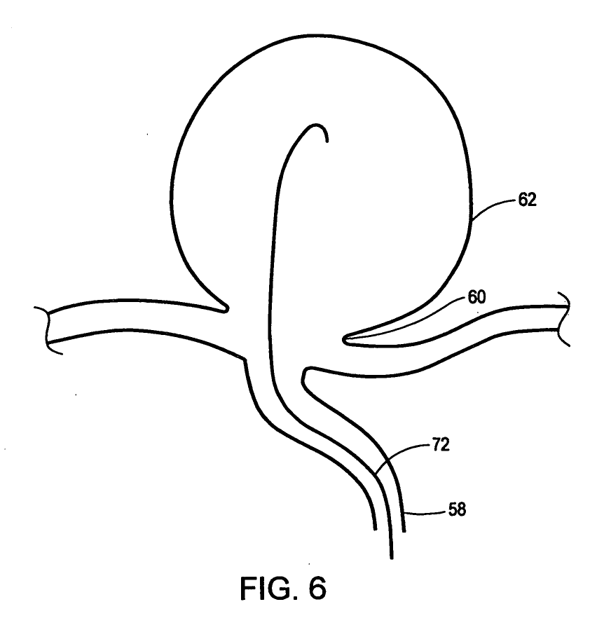

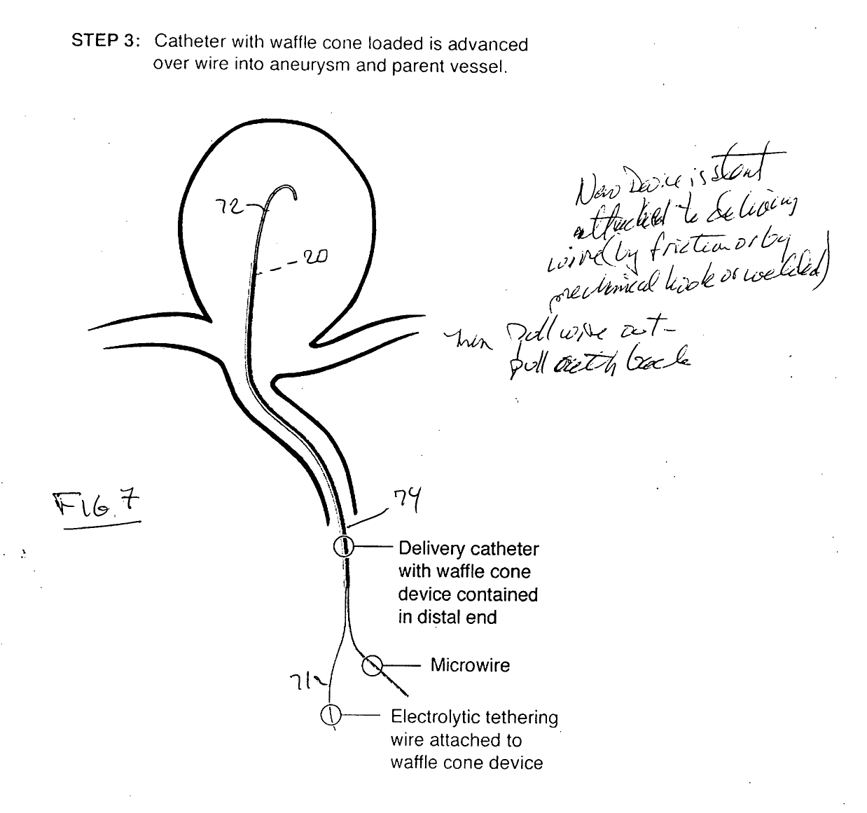

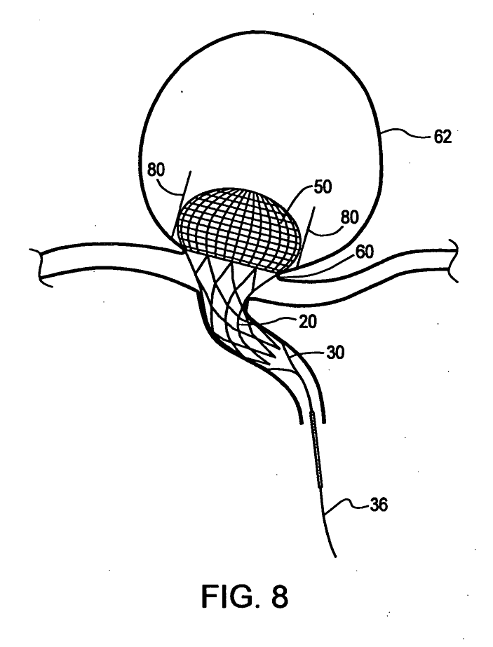

The introduction of an aneurysm treating stent device 20 into an aneurysm is done by threading a microcatheter or sheath 70, bearing a micro wire 72, through the vasculature 58 of the patient and into the bifurcation aneurysm 62, as is represented in FIG. 5. The microcatheter or sheath 70 surrounding the microwire is pulled proximally, so as to leave the bare microwire 72 juxtaposed within the aneurysm 62, as is represented in FIG. 6. A delivery catheter 74 with an unexpanded waffle cone stent device 20 loaded therewithin is threaded over the microwire 72 and that delivery catheter 74 is advanced into the aneurysm through the parent vessel 58 thereadjacent, as is represented in FIG. 7, connected by electrolytic tethering wires 71 which is connected to the proximal end of the waffle cone device 20. The microwire or guidewire 72 is then removed by its withdrawal proximally through the delivery catheter 74. The waffle cone stent device 20 with its attendant distalmost drip chamber thereon, in its unexpanded state, is guided through that parent vessel with the drip chamber disposed nestingly at the neck of the aneurysm 62, as represented in FIG. 8. Withdrawal of the delivery catheter 74 from disposition on the outside of the waffle cone stent 20 and drip chamber 52 permits their respective self-expansion to occur, as represented in FIG. 8. Adjustments in the position of the drip chamber 50 and the waffle cone stent device 20 may be made by the tethering wire 36 which is attached to the proximal most end of the stent device 20, as recited hereinabove. Once the stent device 20 and distally attached drip chamber 50 are properly expanded and emplaced, the tethering wire 36 may be electronically separated from the proximal end of the stent device 20.

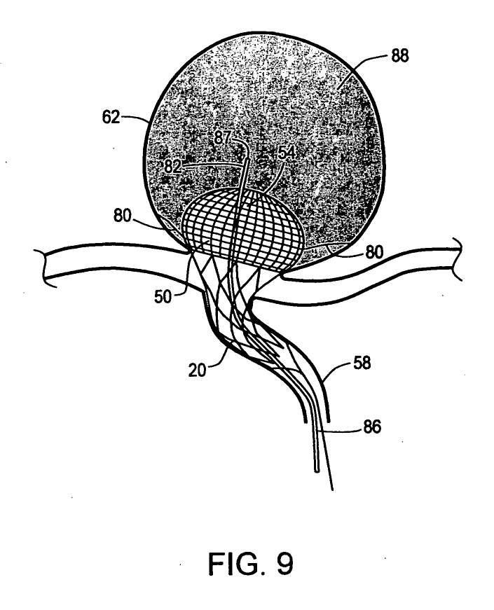

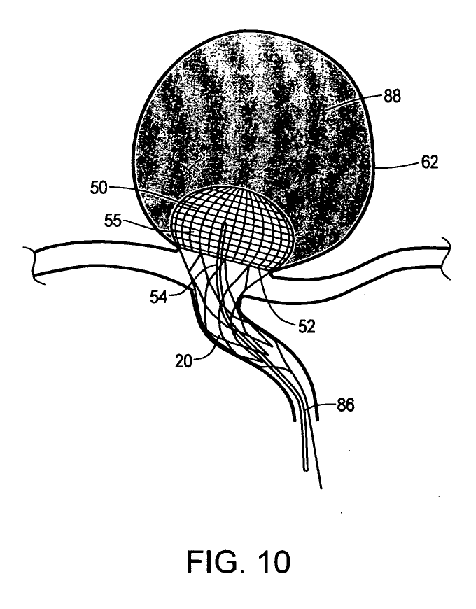

Upon withdrawal of the delivery stent from the drip chamber portions of the stent device 20, in a further preferred embodiment thereof, an arrangement of folded struts 80, shown in FIG. 8, may flare out to their own spring tension or self-expansion capabilities, to permit the drip chamber 50 to be firmly anchored within the neck confines of the aneurysm 62, as represented in FIG. 9.

A new microcatheter or guidewire 82 may be advanced through the waffle cone stent 20 and drip chamber 50 after they has been properly placed within the aneurysm 62, as represented in FIG. 9. That microwire or guide wire 82 would be arranged so as to extend through one of the now expandedly open cells 54 in the floor 52 of the drip chamber 50 and also through the outer cells 55 of the drip chamber 50 as well. A yet further new microcatheter 86 may then be threaded over that microwire or guidewire 82 which extends distally beyond the drip chamber 50, as shown in FIG. 9. Once that new microcatheter 86 is in place distally beyond the distalmost end of the drip chamber 50, that microwire or guidewire 82 is withdrawn proximally therefrom.

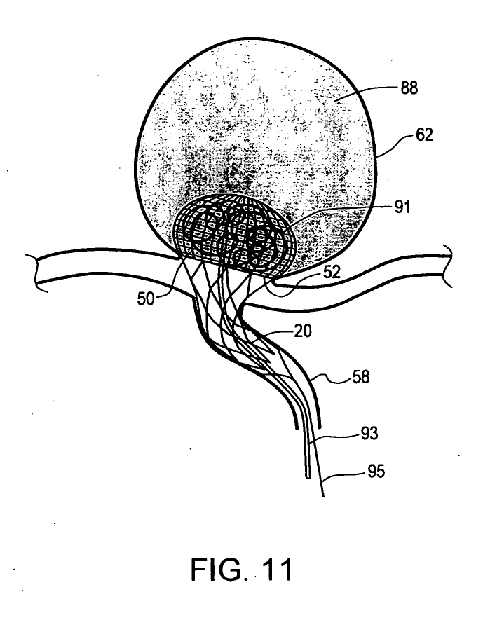

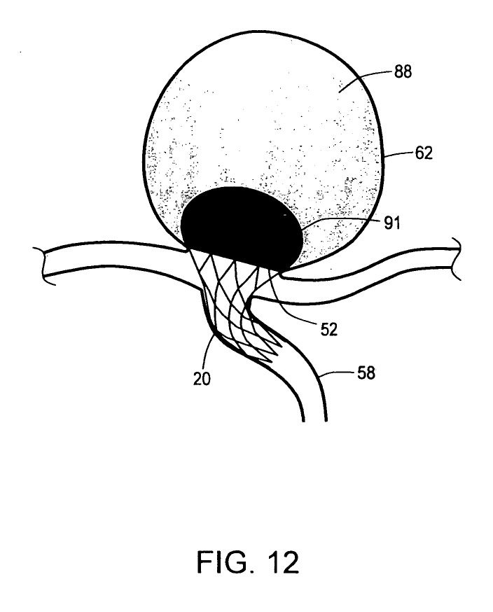

The microcatheter 86 has a distal orifice 87 which then acts as an ejector, through which Onyx™, an embolic agent 88, may be introduced into the aneurysm 62. The dome of the drip chamber 50 having small cellular openings 54 therein, acts as a protective shield to prevent the Onyx 88 from reflux into the parent vessel 58 adjacent the aneurysm 62. Once the Onyx embolic agent 88 has filled the fundus of the aneurysm 62, that delivery catheter 86 is removed. A further microwire or guidewire may then be inserted through the stent device 20 and into the drip chamber 50 through its floor 52. That microwire or guidewire would then be removed and the drip chamber 50 being of a predetermined known volume, may be completely filled with a predetermined amount of a second embolic material, such as a more viscous Onyx, metallic coils, or for example a nitinol plug. The predetermined amount of embolic material thus leaves no voids within the drip chamber 50, minimizing the likelihood of leakage of the initial embolic material into the parent vessel and prevents any undesired collapse or folding of that chamber 50. Once the drip chamber 50 is filled with the second embolic material 91, that drip chamber 50 will thus block the original onyx 88 from entering the parent vessel 58, as represented in FIGS. 11 and 12.

The microcatheters 93 and electrolytic tether wires 95 representatively shown in FIG. 11 may then be removed from the waffle-cone stent device 20 and the aneurysm 62 remains filled with separate volumes of different multiple embolic materials 88 and 91, effectively making the aneurysm 62 generally harmless to the patient. The invention thus comprises a cone-like tapered stent with larger web-like distal openings with smaller proximal openings, and a distalmost chamber which is arranged to expand in the aneurysm, to hold one type embolytic material 88 within the aneurysm 62 and one embolytic material 91 within the chamber 52.

1. A vascular aneurysm treating stent arrangement having a proximal end and a distal end, said stent being formed of a differentially expandable material, wherein said distal end is deformably expandable to a cone shape;
a deformable enclosed chamber arranged on said distal end of said stent.

2. The stent arrangement s recited in claim 1, wherein said deformable chamber has an expandable foraminous floor arranged thereon.

3. The stent arrangement as recited in claim 2, wherein said chamber has wall portions with a smaller opening pattern arranged therethrough.

4. The stent arrangement as recited in claim 1, wherein the stent device is comprised of a woven material.

5. The stent arrangement as recited in claim 1, wherein said stent has a severable tether arranged in its proximal end.

6. A method of treating a bifurcated aneurysm having a neck portion, into a body vessel, comprising:
introducing a stent assembly into said body vessel, said stent having a body portion and a distal chamber on said body portion;
inserting said chamber into said aneurysm;
expanding said chamber to a known dimension and volume.

7. The method as recited in claim 6, including the step of:
introducing a first delivery catheter through said body portion of said stent and through said chamber and into said aneurysm.

8. The method as recited in claim 7, including the step of:
injecting an first embolic material into said aneurysm.

9. The method as recited in claim 8, including the step of:
removing said first delivery catheter from said aneurysm and introducing a second delivery catheter through said body portion of said stent and into said chamber;
injecting a known quantity of a second embolic material into said chamber, completely filling said chamber.

10. The method as recited in claim 6, including the step of:
nesting said chamber within said neck portion of said aneurysm.

11. The method as recited in claim 10, including the step of:
opening a strut arrangement into said aneurysm to secure said chamber within said aneurysm.

12. The method as recited in claim 10, including the step of:
placing a floor in a proximal portion of said chamber to segregate said second embolic material from said body vessel.

13. The method as recited in claim 6, including the step of:
expanding a distal portion of said stent body into a cone shape.

14. The method as recited in claim 13, including the step of:
expanding said chamber into a known volume within said aneurysm simultaneously with said expansion if said stent body.

15. A vascular aneurysm treating stent arrangement having a proximal end and a distal end, said distal end having a larger pattern of openings therethrough than any sidewall openings at said proximal end, upon delivery thereof.

16. The vascular aneurysm treating stent arrangement as recited in claim 15, wherein said distal end of said stent has an expandable web floor disposed thereacross, said floor having an expandable dome-like chamber thereon to permit a first embolytic material to be disposed outwardly thereof, and a second embolytic material to be separately retained within said dome-like chamber.

17. The vascular aneurysm treating stent arrangement as recited in claim 16, wherein said second material comprises metal coils.

18. The vascular aneurysm treating stent arrangement as recited in claim 16, wherein said second material in said chamber comprises a blocking component to said first embolytic material.

19. The vascular aneurysm treating stent arrangement as recited in claim 16, wherein said distal end of said stent has enlarged openings thereacross to permit blood flow across said distal end of said stent, and said distal end of said stent has an expandable, aneurysm-nesting chamber thereon to anchor said stent thereat.

20. The vascular aneurysm treating stent arrangement as recited in claim 19, wherein said distal end of said stent includes a plurality or articulable struts arranged to spread radially outwardly radially adjacent said expandable chamber to further anchor said stent within said aneurysm.
